(19) United States
(12) Patent Application Publication
(10) Pub. No.: US 2010/0222371 A1
(43) Pub. Date: Sep. 2, 2010

(54) PREVENTION OF SURGICAL ADHESIONS

(75) Inventors: Mark Puder, Medfield, MA (US); Arin K. Greene, Wellesley, MA (US)

Correspondence Address: DAVID S. RESNICK, NIXON PEABODY LLP, 100 SUMMER STREET, BOSTON, MA, 02110-2131, US

(73) Assignee: CHILDREN'S MEDICAL CENTER CORPORATION, Boston, MA (US)

(21) Appl. No.: 12/620,665

(22) Filed: Nov. 18, 2009

Related U.S. Application Data
(60) Provisional application No. 61/116,546, filed on Nov. 20, 2008.
(60) Provisional application No. 61/116,860, filed on Nov. 21, 2008.

(51) Int. Cl. A61K 31/517; A61K 31/404; A61K 31/4439; A61K 31/44; A61P 17/02; A61P 17/00; A61P 35/00

(52) U.S. Cl. 514/266.4; 514/414; 514/338; 514/350

(57) ABSTRACT

The present invention relates to methods for treatment (particularly the prevention or suppression) of formation or reformation of adhesions, particularly in the peritoneal or pelvic cavities resulting from wound, surgery, infection, inflammation or trauma. The invention provides methods useful for inhibiting, suppressing or ameliorating adhesion formation in mammals, including humans wherein an individual is administered a compound selected from the group consisting of sunitinib malate, axitinib, semaxanib, sorafenib, ZD1839, and erlotinib. The invention applies to human and veterinary applications. The inventive method has been shown to be especially effective in preventing adhesion formation in the peritoneum following surgery.

### CROSS REFERENCE TO RELATED APPLICATIONS

This application claims the benefit of priority under 35 U.S.C. §119(e) of U.S. Provisional Application No. 61/116,546 filed Nov. 20, 2008 and U.S. Provisional No. 61/116,860 filed Nov. 21, 2008, the contents of which are incorporated herein by reference in its entirety.

### BACKGROUND OF THE INVENTION

Adhesion formation, the joining of two normally separate surfaces due to trauma or inflammation, is a major problem following surgical procedures. Adhesions following surgery frequently cause postoperative pain, blockage of intestines, and infertility. Adhesions are the major cause of intestinal obstruction and it is estimated that following an intra-abdominal procedure, adhesions occur in some 50 to 80 percent of patients. Intestinal obstruction caused by adhesions leads to prolonged hospital stays, additional abdominal surgery, and even death. Abnormal scarring in the abdomen also increases the morbidity of future surgery because adhesions lead to increased blood loss and injury to internal organs. Adhesion formation is also problematic in orthopedic and plastic surgeries, such as in the hand, where impediment of movement is frequently troublesome to the patient.

The mechanism of surgical adhesion formation can be summarized as follows: a trauma to a tissue is rapidly followed by an inflammatory reaction; exudation of plasma, and deposition of a fibrin matrix. Subsequently the lesion is healed by the degradation of the fibrin deposition, and by proliferation of the mesenchymal lining of the peritoneum. If the repair process is not completed within a few days, fibroblast proliferation starts which ultimately will end in collagen deposition and adhesion formation. Key mediators of this process include fibrin and fibrinolysis, macrophages and their secretion products such as growth hormones and cytokines, as well as the epithelial repair process. This repair process results in adhesion formation, which is largely independent from the extent of the trauma.

Prevention of adhesion formation has been attempted mechanically to modulate the inflammatory reaction. Mechanical adhesion prevention has been performed with barrier methods and by the instillation of viscous fluids at the end of surgery in an attempt to keep opposing surfaces separated. Recently, coating adjoining surfaces with biodegradable gels has been explored.

These known approaches have been only moderately successful and more effective methods of prevention are necessary.

### SUMMARY OF THE INVENTION

The present invention provides a method for the minimization or prevention of adhesion formation during or following a surgical procedure. The method comprises administering to the patient in need thereof a therapeutically effective amount of a compound selected from the group consisting of sunitinib malate, axitinib, semaxanib, sorafenib, ZD1839, and erlotinib.

SU11248 (sunitinib malate; sunitinib) is available commercially from Pfizer Inc. under the trade name SUTENT®. Axitinib (AG013736) can also be obtained from Pfizer Inc., while sorafenib (BAY 43-9006) can be obtained commercially from Bayer AG. ZD1839 (Beringer; genfitinib) can be purchased from AstraZeneca PLC under the trade name IRESSA®. Erlotinib (OSI-774) can be obtained from Genentech or OSI Pharmaceuticals in the United States and from Roche under the trade name TARCEVA®. Semaxanib (SU 5416) is currently not available on the market but was originally developed by Pharmacia Corporation.

In one embodiment, the adhesion is between organ surfaces. As used herein, the term “organ surface” is intended to encompass any internal organ or tissue of a living animal including but not limited to the uterus, intestine, peritoneum, omentum, stomach, liver, kidneys, heart, and lung.

The present invention also provides a method of treating, preventing, or minimizing keloid formation, implant contractures including, but not limited to synthetic autologous, or heterologous implants such as used in breast, abdominal wall, cosmetic, orthopedic, hand, craniofacial or urologic implants, benign hypertrophic tumors, or scarring related to incision or cosmetic surgery. These methods comprise administering to a patient a therapeutically effective amount of a compound selected from the group consisting of sunitinib malate, axitinib, semaxanib, sorafenib, ZD1839, and erlotinib.

As used herein the term “comprising” or “comprises” is used in reference to compositions, methods, and respective component(s) thereof, that are essential to the invention, yet open to the inclusion of unspecified elements, whether essential or not.

As used herein the term “consisting essentially of” refers to those elements required for a given embodiment. The term permits the presence of elements that do not materially affect the basic and novel or functional characteristic(s) of that embodiment of the invention.

The term “consisting of” refers to compositions, methods, and respective components thereof as described herein, which are exclusive of any element not recited in that description of the embodiment.

As used in this specification and the appended claims, the singular forms “a,” “an,” and “the” include plural references unless the context clearly dictates otherwise. Thus for example, references to “the method” includes one or more methods, and/or steps of the type described herein and/or which will become apparent to those persons skilled in the art upon reading this disclosure and so forth.

### BRIEF DESCRIPTION OF THE DRAWINGS

FIG. 1. Cecum (A) lifted from abdominal cavity adhesion free in sunitinib treated animal (left). Cecum (A) adhered to itself with omental adhesion (B) (right).

FIG. 2. Effect of sunitinib on intra-abdominal adhesion formation. Sunitinib significantly decreased intra-abdominal adhesion formation (i) (P=0.002).

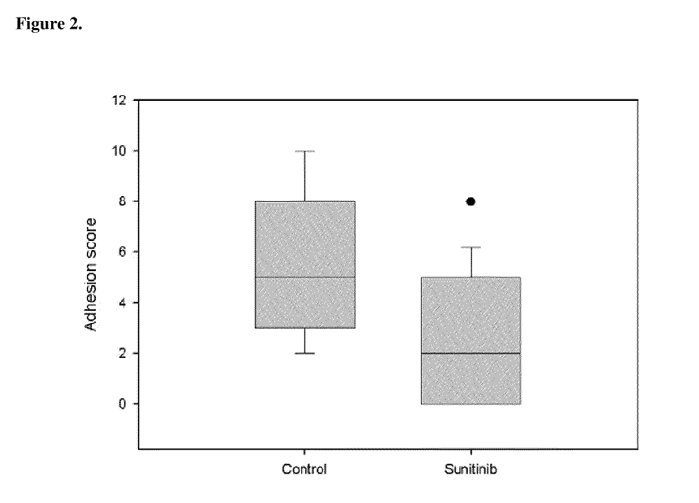

FIG. 3. Long-term effect of sunitinib on intra-abdominal adhesion formation. Sunitinib significantly decreased intra-abdominal adhesion formation (i) (P=0.049).

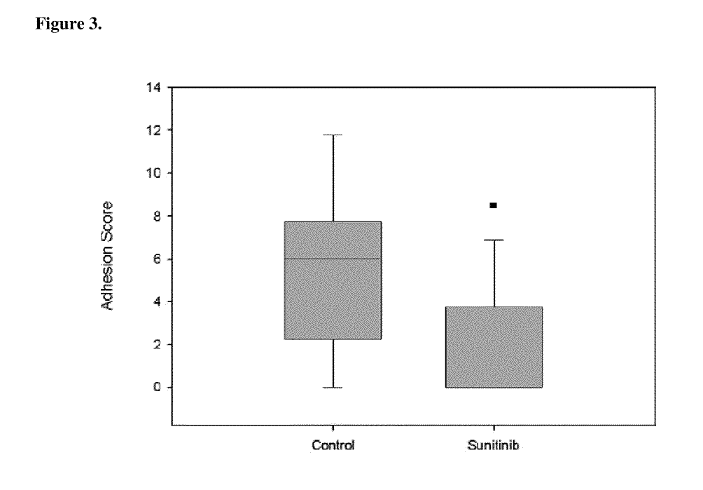

FIG. 4. is a micrograph indicating that a rabbit treated with sunitinib is free from abdominal cavity adhesions.

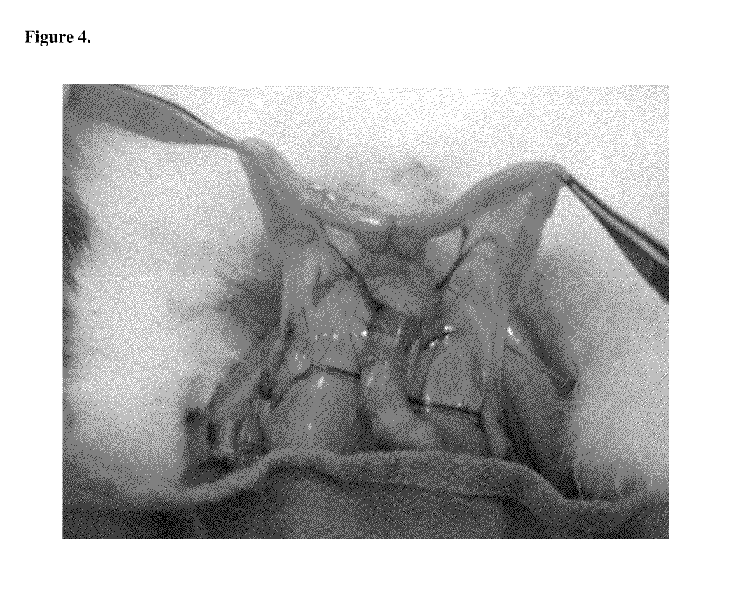

FIG. 5. is a micrograph indicating an intra-abdominal adhesion that is present in a control-treated rabbit.

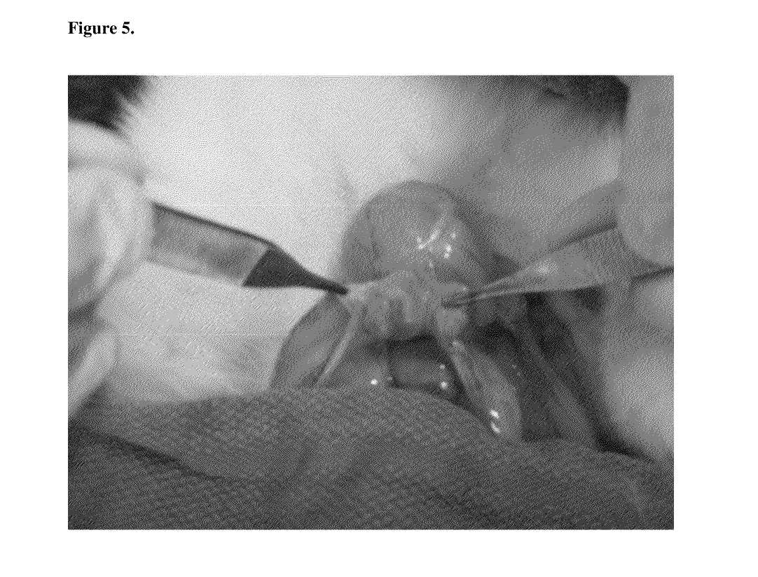

Table 1 shows a study where either SU11248 or vector only control were given to 7 week old male C57BL/6 mice in order to assess adhesion prevention. SUI 1248 significantly prevention adhesion formation.

Table 2. shows an exemplary adhesion scoring criteria as used in Example 2.

Table 3. shows adhesion scores of control versus sunitinib-treated mice.

Table 4. shows long-term adhesion scores of control versus sunitinib-treated mice (post-operative day 30).

Table 5. Adhesion scores of rabbits treated with sunitinib vs. controls.

### DETAILED DESCRIPTION OF THE INVENTION

The inventors have discovered methods of treating, preventing, and minimizing post-operative adhesion formation. The method comprises administering to a patient in need thereof a therapeutically effective amount of a compound selected from the group consisting of sunitinib (also referred to herein as SU11248; sunitinib malate; SUTENT®) axitinib (also referred to herein as AG013736), semaxanib (also referred to herein as SU5416), sorafenib (also referred to herein as BAY 43-9006), ZD1839 (also referred to herein as IRESSA®, and erlotinib (also referred to herein as TARCEVA®, or OSI-774).

The desired compound may be administered in combination with other therapies for minimizing adhesion formation.

This invention relates to methods for treatment (particularly the prevention or suppression) of formation or reformation of adhesions, particularly in the peritoneal or pelvic cavities resulting from wound, surgery, infection, inflammation or trauma. The invention provides methods useful for inhibiting, suppressing or ameliorating adhesion formation in mammals, including humans. The invention applies to human and veterinary applications. The inventive method has been shown to be especially effective in preventing adhesion formation in the peritoneum following surgery. In addition, the present invention finds utility in other contexts, e.g., for cardiovascular, orthopedic, thoracic, ophthalmic, CNS and other uses, where prevention of the formation of adhesions is a significant concern.

The term “adhesion formation” as used herein in its medical sense refers to conglutination, the process of adhering or uniting of two surfaces or parts. For example, the union of the opposing surfaces of a wound, or opposing surfaces of peritoneum. Also, adhesions, in the plural, can refer to inflammatory bands that connect opposing serous surfaces. The term adhesion, as used herein, also includes fibrinous adhesions, which are adhesions that consist of fine threads of fibrin resulting from an exudate of plasma or lymph, or an extravasion of blood. Keloid, a smooth overgrowth of fibroblastic tissue that arises in an area of injury or, occasionally, spontaneously is also a form of adhesion. Basal adhesion formation as used herein in its medical sense is the basal level of adhesion formation that occurs after injury wounding (e.g. surgery) exposed to an atmosphere which contains sufficient oxygen to avoid a condition of hypoxia or of hyperoxia. The methods of the present invention are useful in the treatment of all such adhesions.

A preferred embodiment of the present invention shows that adhesion formation is significantly suppressed by the administration of sunitinib. In addition, a compound selected from the group consisting of sunitinib malate, axitinib, semaxanib, sorafenib, ZD1839, and erlotinib, is contemplated for use in the manufacture of a medicament for treatment of (post-operative) adhesion formation.

The present invention can also be applied to the treatment, prevention, or minimization of keloid formation, implant contractures such as breast, benign hypertrophic tumors, and scarring in cosmetic or incision surgery by administering sunitinib.

A compound can be administered to the patient before, during, or after surgery or any combination thereof. In general, administration should be 12-48 hours prior to the time of surgery and for at least 24-48 hours post-surgery. Alternatively, the desired compound is administered 72 hours prior to surgery and treatment is continued up to 2 weeks after surgery. When administered after abdominal surgery, the preferred method of administration is by suppository.

In one embodiment the adhesion is between organ surfaces. As used herein, the term “organ surface” is intended to encompass any internal organ or tissue of a living animal including but not limited to the uterus, intestine, peritoneum, omentum, stomach, liver, kidneys, heart, and lung.

The present invention also provides a method of treating, preventing, or minimizing keloid formation, implant contractures including, but not limited to synthetic autologous, or heterologous implants such as used in breast, abdominal wall, cosmetic, orthopedic, hand, craniofacial or urologic implants, benign hypertrophic tumors, or scarring related to incision or cosmetic surgery. These methods comprise administering to a patient a therapeutically effective amount of a compound selected from the group consisting of sunitinib malate, axitinib, semaxanib, sorafenib, ZD1839, and erlotinib. The desired compound can be administered before, during, or after treatment.

In one embodiment, a composition of the present invention comprises a compound selected from the group consisting of sunitinib malate, axitinib, semaxanib, sorafenib, ZD1839, and erlotinib in combination with a pharmaceutically acceptable carrier. Suitable formulations include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, for example in the range of in one embodiment about 0.1 to 10 mg/ml, in another embodiment about 2.0 mg/ml; and/or mannitol or another sugar, for example in the range of in one embodiment 10 to 100 mg/ml, in another embodiment about 30 mg/ml; phosphate-buffered saline (PBS), and any other formulation agents conventional in the art.

Dosage amount and interval may be adjusted individually to provide plasma levels of the active compound which are sufficient to maintain inhibitory effects. Usual patient dosages for systemic administration range from 1-2000 mg/day, commonly 1-250 mg/day, and typically from 10-150 mg/day.

Suitable methods for administration of a composition of the present invention include but are not limited to intravascular, subcutaneous, or intratumoral administration. For delivery of compositions to pulmonary pathways, compositions can be administered as an aerosol or coarse spray. In a preferred embodiment of the invention, direct administration on the site of the expected adhesion is employed. In one embodiment of the methods described herein, the composition is administered using a suppository.

In one embodiment, a therapeutically effective amount of a composition of the invention is administered to a subject. A “therapeutically effective amount” is an amount of a composition comprising e.g., sunitinib sufficient to produce a measurable anti-adhesion response. Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, expected adhesion size and longevity, and the physical condition and prior medical history of the subject being treated. Determination and adjustment of a therapeutically effective dose, as well as evaluation of when and how to make such adjustments, are known to those of ordinary skill in the art of medicine.

In one embodiment of the methods described herein, a minimally therapeutic dose of sunitinib is administered. The term “minimally therapeutic dose” refers to the smallest dose, or smallest range of doses, determined to be a therapeutically effective amount as that term is used herein.

In a specific embodiment it should be clear that the therapeutic method of the present invention for the treatment of suppression of adhesion formation can also be used in combination with any other therapy known in the art for the treatment of post operative adhesion formation. For example, sunitinib for preventing adhesion formation is preferably administered in conjunction with a drug delivery system which maintains an effective concentration of the compound at a site of potential adhesion formation during the perioperative interval. A desired compound for use in treatment of suppressing and/or minimizing adhesion formation can be administered as the primary therapeutic agent or can be co-administered with one or more additional therapeutic agents. It is also contemplated herein that more than one compound selected from the group consisting of sunitinib malate, axitinib, semaxanib, sorafenib, ZD1839, and erlotinib are used in combination for the treatment of surgical adhesions.

Sunitinib (also referred to herein as sunitinib malate) can be obtained from Pfizer Inc. under the trade name SUTENT®, and is described in U.S. Pat. No. 6,573,293, which is incorporated herein in its entirety. Axitinib (AG013736) can also be obtained from Pfizer Inc., and is described in e.g., U.S. Pat. No. 6,531,491, which is incorporated herein by reference in its entirety. Sorafenib (BAY 43-9006) can be obtained commercially from Bayer AG, and is described in eg., U.S. Pat. No. 7,235,576, which is incorporated herein by reference in its entirety. ZD1839 (Beringer; genfitinib) can be purchased from AstraZeneca PLC under the trade name IRESSA®, and is described in e.g., U.S. Pat. No. 5,457,105, which is incorporated herein in its entirety. Erlotinib (OSI-774) can be obtained from Genentech or OSI Pharmaceuticals in the United States and from Roche under the trade name TARCEVA®. TARCEVA® is described in e.g., U.S. Pat. No. 5,747,498, which is herein incorporated by reference in its entirety. Semaxanib (SU 5416) is currently not available on the market but was originally developed by Pharmacia Corporation and is described in U.S. Pat. No. 5,792,783, which is herein incorporated by reference in its entirety.

It is understood that the foregoing detailed description and the following examples are illustrative only and are not to be taken as limitations upon the scope of the invention. Various changes and modifications to the disclosed embodiments, which will be apparent to those of skill in the art, may be made without departing from the spirit and scope of the present invention. Further, all patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents are based on the information available to the applicants and do not constitute any admission as to the correctness of the dates or contents of these documents.

The present invention may be as described in any one of the following numbered paragraphs.

1. A method of treating, preventing, or minimizing adhesion formation during or following a surgical procedure comprising administering to a patient in need thereof a therapeutically effective amount of a compound selected from the group consisting of sunitinib malate, axitinib, semaxanib, sorafenib, ZD1839, and erlotinib.
2. A method of treating, preventing, or minimizing keloid formation during or following a surgical procedure comprising administering to a patient in need thereof a therapeutically effective amount of a compound selected from the group consisting of sunitinib malate, axitinib, semaxanib, sorafenib, ZD1839, and erlotinib.
3. A method of treating or preventing benign hypertrophic tumors comprising administering to a patient in need thereof a therapeutically effective amount of a compound selected from the group consisting of sunitinib malate, axitinib, semaxanib, sorafenib, ZD1839, and erlotinib.
4. A method of treating, preventing, or minimizing scar formation during or following a surgical procedure comprising administering to a patient in need thereof a therapeutically effective amount of a compound selected from the group consisting of sunitinib malate, axitinib, semaxanib, sorafenib, ZD1839, and erlotinib.
5. A method of treating, preventing, or minimizing contracture formation during or following implantation of synthetic, autologous, or heterologous implants comprising administering to a patient in need thereof a therapeutically effective amount of a compound selected from the group consisting of sunitinib malate, axitinib, semaxanib, sorafenib, ZD1839, and erlotinib.
6. The method of paragraph 1 wherein the adhesion is between organ or tissue surfaces.
7. The method of paragraph 4 wherein the surgical procedure is cosmetic or by incision.
8. The method of paragraph 5 wherein the implant is a breast implant, implant used in cosmetic surgery, abdominal implant, urologic implant, or an orthopedic implant.
9. The method of any one of paragraphs 1, 2, 3, 4, or 5 wherein the compound is administered to the patient before surgery.
10. The method of any one of paragraphs 1, 2, 3, 4, or 5 wherein the compound is administered by applying to organ surfaces during surgery.
11. The method of any one of paragraphs 1, 2, 3, 4, or 5 wherein the compound is administered after surgery.
12. The method of paragraph 1 wherein the compound is administered by use of a suppository.

### EXAMPLES

### Example 1

Ten 7 week old male C57BL/6 mice were anesthetized with isoflurane and a midline laparotomy was performed. A 5.0 mm×5.0 mm piece of sterile silicone was attached to the right abdominal wall with 7.0 prolene sutures. The cecum then was gently rubbed with 2 cotton swabs to promote adhesion formation.

Two groups of 5 mice were treated with either 40 mg/kg/day of sunitinib in methylcellulose or methyl cellulose alone beginning the day before the adhesion operation and administered daily for 10 days. After 10 days of treatment, the animals were anesthetized with avertin, sacrificed, and the adhesions were scored.

The total adhesion score was the sum of the 3 individual scores. A minimum score was 0 and the maximum score was 12. Tenacity was rated as none (0), adhesions fell apart (1), lysed with traction (2), lysed with blunt dissection (3), and lysed with sharp dissection (4). Extent was measured as the percent of the patch covered by adhesion: 0% (0), <25% (1), 25 to 50% (2), 50 to 75% (3), and >75% (4). Cecal adhesions were graded as none (0), adhesions fell apart (1), lysed with traction (2), lysed with blunt dissection (3), and lysed with sharp dissection (4). The differences between the treated and untreated groups were significant (P=0.021). The results are shown herein in Table 1.

### Example 2

### Methods

Thirty-seven 6- to 8-wk old C57BLJ6 mice (The Jackson Laboratory, Bar Harbor, Me.) were housed in cages of five mice with food and water ad libitum. The mice were acclimated to the facility for at least 72 h prior to surgery. A standard mouse adhesion model was performed as previously described (Chiang S C., et al (2000) J Pediatr Surg 35:189). After the mice were anesthetized with inhaled isoflurane, a midline incision was made, the cecum was eviscerated, rubbed gently with two cotton swabs, and then returned to anatomical position. A silicone patch (5.0×5.0×0.127 mm) (Bentec Medical, Woodland, Calif.) was sutured to the right abdominal wall, lateral to the epigastric vessels, with 7-0 nylon. One day prior to surgery, the mice were randomly treated with sunitinib (40 mg/kg in 100 methylcellulose) or methylcellulose control (100 uL). This dose was selected based on previous studies (Morimoto A M., (2004) Oncogene 23:1618; Takahashi H., et al (2006) J Ocul Pharmacol Ther 22:213; Pietras K., et al (2005) J Clin Ocol 23:939) Both groups were treated daily by orogastric gavage at the same dose for 10 days. On postoperative day 10, the mice were sacrificed and adhesions were scored by an observer who was blinded to the treatment groups, based on a previously described scoring system (Chiang S C., et al (2000), supra). The experiment was then repeated with sacrifice on postoperative day 30. The adhesion score was based on the extent of the patch covered by adhesions, the tenacity of adhesions to the patch, and the strength of cecal adhesions. Each criterion was scored from 0-4 and the sum of the three scores was the final adhesion score. The lowest and highest possible scores were 0 and 12, respectively. The tenacity of the adhesions to the patch was scored: 0=none, 1=adhesions fell apart, 2=lysed with traction, 3=lysed with blunt dissection, 4=lysed with sharp dissection. The extent of the patch covered was scored: 0=0%, 1=<25%, 2=25% to 50%, 3=50% to 75%, 4=>75%. The tenacity of cecal adhesions was scored: 0=none, 1=adhesions fell apart, 2=lysed with traction, 3=lysed with blunt dissection, 4=lysed with sharp dissection. Statistical analysis of adhesion scores was conducted using the Mann-Whitney rank sum test. The conventional 2-tailed P-value of 0.05 was used to establish significance. The results are presented as median adhesion score and interquartile range (IQR: 25th-75th percentile). The statistical program used was SigmaStat (SPSS, Chicago, Ill.).

### Results

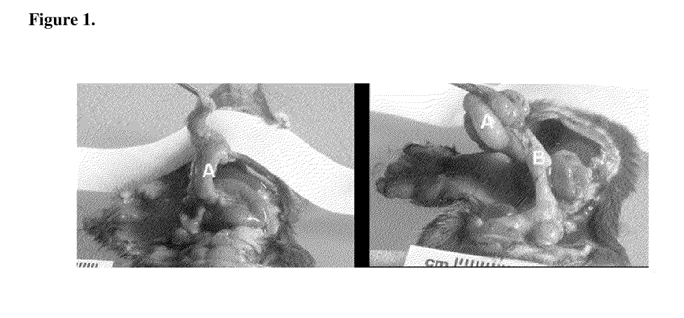

The adhesion model was performed on 37 mice. There were no intraoperative complications. All 19 control mice developed intra-abdominal adhesions. Six of the eighteen (33.3%) mice in the treatment group were completely adhesion-free (FIG. 1). The control mice had a median tenacity, extent, and cecal adhesion score of 1.0 (IQR 0.0-2.8), 1.0 (IQR 0.0-4.0), and 3.0 (IQR 2.0-3.0), respectively. The sunitinib treated mice had a median tenacity, extent, and cecal adhesion score of 0.0 (IQR 0.0-1.0), 0.0 (IQR 0-2.0), and 2.0 (IQR 0-2.0), respectively. In comparing the treatment and control groups, the P-values for tenacity, extent, and cecal adhesions were 0.68, 0.91, and 0.002, respectively. The total median adhesion score for the control group was 5.0 (IQR 3.0-8.0) and for the sunitinib treated group was 2.0 (IQR 0.0-5.0) (P=0.002). Intraabdominal adhesions were significantly decreased in animals treated with sunitinib in comparison to the control group, with P=0.002.

The adhesion model was then repeated to determine the long-term effect of sunitinib on intra-abdominal adhesions. There were no intraoperative complications. Mice were treated for 10 d, starting the day prior to surgery with either sunitinib or methylcellulose control, and were sacrificed on postoperative day 30. At the time of sacrifice, six of the ten (60%) mice in the treatment group were completely adhesion-free. The control mice had a median tenacity, extent, and cecal adhesion score of 2.0 (IQR 0.0-2.0), 1.5 (IQR 1.0-2.0), and 2.0 (IQR 0.0-3.0), respectively. The sunitinib treated mice had a median tenacity, extent, and cecal adhesion score of 0.0 (IQR 0.0-0.5), 0.0 (IQR 0-1.0), and 0.0 (IQR 0-2.0), respectively. In comparing the treatment and control groups, the P-values for tenacity, extent, and cecal adhesions were 0.011, 0.049, and 0.225, respectively. The total median adhesion score for the control group was 6.0 (IQR 3.0-7.0) and for the sunitinib treated group was 0.0 (IQR 0.0-3.0) (P=0.049). Long-term results were consistent with those seen at postoperative day 10. Intraabdominal adhesions were decreased in animals treated with sunitinib in comparison with the control group, with P=0.049.

Intra-abdominal adhesions most commonly occur following operative procedures, although they may also be the result of trauma, intra-abdominal sepsis, radiation, endometriosis, and pelvic inflammatory disease. Minimal bowel manipulation, hemostasis, and powderfree gloves are strategies that may minimize adhesion formation (Winston R M., et al. (1991) Br J Obstet Gynaecol 98:637; Risberg B. (1997) Eur J Surg Suppl (577):32) Various adjuvant techniques and pharmacological agents for adhesion prevention have been tested with mixed results. For example, instillation of fluids intraperitoneally has been used to keep deperitonealized surfaces separated (Rose B I., et al (1991) J Reprod Med 36:537; Verco S J., et al (2000) Hum Reprod 15:764). However, the use of intra-abdominal crystalloids, dextran, and icodextrin have not been shown to be effective (Wiseman D M., (1998) Fertil Steril 70:702; Metwqally M., et al (2006) Cochrane Database Syst Rev DC001298; diZerega G S., et al. (2002) Hum Reprod 17:1031).

Adhesion prevention using pharmacologic agents also has been tested with variable efficacy. Some studies have demonstrated that nonsteroidal anti-inflammatory drugs block the inflammatory response and thus prevent adhesions, while others studies have been unable to show a protective effect (Siegler A M., et al (1980), supra; Holtz G. (1982) Fertil Steril 37:582; Luciano A A., et al (1983) Am J Obstet Gynecol 146(88)). A review by the Cochrane collaboration concluded that there was no significant evidence in the literature to support the use of steroids for adhesion prevention after gynecologic surgery (Watson A., et al (2000) Cochrane Database Syst Rev CD001298). Additionally, some studies have focused on up-regulation of fibrinolysis as a mechanism for adhesion prevention. Hellebreker et al. outlined the results of many animal studies and a few human studies on the use of fibrinolytics, including streptokinase and recombinant human tissue plasminogen activating factor, in postoperative adhesion prevention (Hellebrekers B W., et al (2000) Fertil Steril 74:203). Although most of the animal studies demonstrated a decrease in adhesions, no definitive conclusions could be made from the human studies. More recently, statins, which may decrease adhesion formation through stimulation of fibrinolysis, have also been tested with encouraging preliminary animal results (Aarons C G., et al (2007) Ann Surg 245:176). Greene et al. suggested that the observed efficacy of selective cyclooxygenase-2 inhibitors on the prevention of intra-abdominal adhesions in a murine model was through an antiangiogenic mechanism (Greene A K., et al (2005) Ann Surg 242:140).

In this study, mice treated with sunitinib not only had fewer adhesions than control animals, but six of 18 (33.3%) mice in the treatment group were completely adhesion-free on postoperative day 10, and six of 10 (60%) were completely adhesion-free on postoperative day 30, indicating that antiangiogenic drugs may prove efficacious for the treatment of adhesions clinically.

### Example 3

Twenty female New Zealand white rabbits underwent a standard rabbit adhesion procedure. The uterus and its bilateral horns were eviscerated, and abraded with the scalpel until punctate hemorrhages appeared. Collateral blood supply in the right mesosalpinx was tied off with 5-0 silk ties. The uterus was then returned to normal anatomic position. One day prior to surgery, the rabbits were randomly assigned to be treated with sunitinib at a dose of 10 mg/kg or saline control via orogastric gavage. Both groups were treated daily for a total of ten days. On postoperative day ten, the rabbits were sacrificed and their adhesions were scored by two observers who were blinded to their treatments. Adhesions are graded based on their tenacity (0=no adhesions, 1=adhesions fall apart, 2=adhesions lysed by traction, 3=adhesions lysed with blunt dissection, 4=adhesions lysed with sharp dissection) and the percent of uterus involved (0=none, 1=<25%, 2=25-50%, 3=51-75%, 4=>75%).

Nine out of the ten control rabbits developed adhesions. Two rabbits in the sunitinib treated group died from perforation into the mediastinum during the gavaging process. Three of the eight surviving rabbits in the sunitinib treated group were completely adhesion free. The sunitinib treated rabbits had a median tenacity score of 1.0 [inter-quartile range (IQR) 0-1.75; range 0-2] compared to a score of 3.25 (IQR 3-3.5; range 0-4) in the control animals (p=0.004). Median percent involvement score for the sunitinib treated rabbits vs. controls was 1.0 (IQR 0-1.0; range 0-1) and 4.0 (IQR 4.0-4.0; range 0-4) respectively (p=0.003). Collectively, the sunitinib treated rabbits had a median total adhesion score of 2 (IQR 0-2.75; range 0-3) compared to 7 (IQR 6-7.5; range 0-8) in the control group (p=0.003).

All references described herein are incorporated herein by reference in their entirety.

TABLE 1
SU11248 VEGF II receptor antagonist vs control in adhesion
prevention in mice.

| | | | Cecal | | |
|---|---|---|---|---|---|
| | Tenacity | Extent | adhesions | Total | comments |
| CONTROL | | | | | |
| 1 | 0 | 0 | 3 | 3 | |
| 2 | 3 | 4 | 2 | 9 | Patch covered w/ |
| | | | | | omentum |
| | | | | | and SB |
| 3 | 3 | 4 | 1 | 8 | Fluid filled cecum |
| | | | | | stuck to |
| | | | | | patch, ileus |
| 4 | 3 | 4 | 2 | 9 | |
| 5 | 2 | 4 | 2 | 8 | |
| SU11248 | | | | | |
| 1 | 1 | 3 | 2 | 6 | Looked good |
| | | | | | overall |
| 2 | 0 | 0 | 2 | 2 | |
| 3 | 0 | 0 | 0 | 0 | |
| 4 | 0 | 0 | 1 | 1 | |
| 5 | 2 | 3 | 0 | 5 | |

TABLE 2
Adhesion Scoring

```
Tenacity (of adhesion to patch):
  None (0)
  Adhesions fell apart (1)
  Lysed with traction (2)
  Lysed with blunt dissection (3)
  Lysed with sharp dissection (4)
Extent (percent of patch covered by adhesion):
  0% (0)
  <25% (1)
  25-50% (2)
  50-75% (3)
  >75% (4)
Cecal adhesion tenacity:
  None (0)
  Adhesions fell apart (1)
  Lysed with traction (2)
  Lysed with blunt dissection (3)
  Lysed with sharp dissection (4)
```

TABLE 3
Adhesion Scores of Control versus Sunitinib Treated Mice

| | Median tenacity | Median extent | Median cecal | Total score | Adhesion free mice |
|---|---|---|---|---|---|
| Control (n = 19) | 1.0 (IQR 0-2.8) | 1.0 (IQR 0-4.0) | 3.0 (IQR 2.0-3.0) | 5.0 (IQR 3.0-8.0) | 0 |
| Sunitinib (n = 18) | 0 (IQR 0-1.0) | 0 (IQR 0-2.0) | 2.0 (IQR 0-2.0) | 2.0 (IQR 0-5.0) | 6 |
| P* | 0.068 | 0.091 | 0.002 | 0.002 | NA |

Note.
IQR indicates interquartile range (between 25th and 75th percentile);
NA = not applicable.
*Statistical analysis comparing adhesion scores using Mann-Whitney rank sum test.
P < 0.05 is considered statistically significant.

TABLE 4
Long-term Adhesion Scores of Control Versus Sunitinib Treated Mice (Post-operative Day 30)

| | Median tenacity | Median extent | Median cecal | Total score | Adhesion free mice |
|---|---|---|---|---|---|
| Control (n = 10) | 2.0 (IQR 0-2.0) | 1.5 (IQR 0-2.0) | 2.0 (IQR 0.0-3.0) | 6.0 (IQR 3.0-7.0) | 2 |
| Sunitinib (n = 10) | 0 (IQR 0-0.5) | 0 (IQR 0-1.0) | 0 (IQR 0-2.0) | 0 (IQR 0-3.0) | 6 |
| P* | 0.011 | 0.049 | 0.225 | 0.049 | NA |

Note.
IQR indicates interquartile range (between 25^{th} and 75^{th} percentile);
NA = not applicable.
*Statistical analysis comparing adhesion scores using Mann-Whitney Rank Sum test.
P < 0.05 is considered statistically significant.

TABLE 5

| | | | | Adhesion |
|---|---|---|---|---|
| | Tenacity to | Extent of Uterus | | Free |
| | Uterus | Covered | Total Score | Rabbits |
| Saline alone | 3.25 | 4.0 | 7.0 | 1 |
| (N = 10) | (3.0-3.5) | (4.0-4.0) | (6.0-7.5) | |
| Sunitinib + | 1.0 | 1.0 | 2.0 | 3 |
| saline | (0-1.75) | (0-1.0) | (0-2.75) | |
| (N = 8) | | | | |
| p-value* | 0.004 | 0.003 | 0.003 | |

Values reported as median (IQR)
*Mann-Whitney Rank Sum test

1. A method of treating, preventing, or minimizing adhesion formation during or following a surgical procedure comprising administering to a patient in need thereof a therapeutically effective amount of a compound selected from the group consisting of sunitinib malate, axitinib, semaxanib, sorafenib, ZD1839, and erlotinib.

2. A method of treating or preventing benign hypertrophic tumors comprising administering to a patient in need thereof a therapeutically effective amount of a compound selected from the group consisting of sunitinib malate, axitinib, semaxanib, sorafenib, ZD1839, and erlotinib.

3. A method of treating, preventing, or minimizing scar formation during or following a surgical procedure comprising administering to a patient in need thereof a therapeutically effective amount of a compound selected from the group consisting of sunitinib malate, axitinib, semaxanib, sorafenib, ZD1839, and erlotinib.

4. The method of claim 1, wherein the adhesion is between organ or tissue surfaces.

5. The method of claim 3, wherein the scar comprises a keloid formation.

6. The method of claim 1, wherein the surgical procedure is cosmetic or by incision.

7. The method of claim 6, wherein the surgical procedure comprises implanting a breast implant, an implant used in cosmetic surgery, an abdominal implant, a urologic implant, or an orthopedic implant.

8. The method of claim 1, wherein said compound is administered to said patient before surgery.

9. The method of claim 1, wherein said compound is administered by applying to organ surfaces during surgery.

10. The method of claim 1, wherein said compound is administered after surgery.

11. The method of claim 1, wherein said compound is administered by use of a suppository.
